(19) United States
(12) Patent Application Publication
(10) Pub. No.: US 2011/0207216 A1
(43) Pub. Date: Aug. 25, 2011

(54) Synthetic Peptide (Meth) Acrylate Microcarriers

(76) Inventors: Arthur Winston Martin, Horseheads, NY (US); Kevin Robert McCarthy, Horseheads, NY (US); Shawn Michael O'Malley, Horseheads, NY (US); Simon Kelly Shannon, Horseheads, NY (US); Meenal Pauddar Soni, Painted Post, NY (US)

(21) Appl. No.: 13/028,635

(22) Filed: Feb. 16, 2011

Related U.S. Application Data
(60) Provisional application No. 61/308,130, filed on Feb. 25, 2010.

(51) Int. Cl. C12N 5/0775

(52) U.S. Cl. 435/372; 435/404

(57) ABSTRACT

A process for forming microcarriers includes contacting an initiator-conjugated microcarrier base with one or more monomers and activating the initiator to initiate polymerization and to graft a polymer from the base via the initiator or a remnant thereof. At least one of the monomers is conjugated to a polypeptide so that incorporation of the monomer into the forming polymer conjugates the polypeptide to the polymeric coating as it is formed in situ.

### CLAIMING BENEFIT OF PRIOR FILED U.S. APPLICATION

This application claims the benefit of U.S. Provisional Application Ser. No. 61/308,130, filed on Feb. 25, 2010. The content of this document and the entire disclosure of publications, patents, and patent documents mentioned herein are incorporated by reference.

### FIELD

The present disclosure relates to cell culture microcarriers, and more particularly to synthetic, chemically-defined microcarriers.

### SEQUENCE LISTING

This application contains a Sequence Listing electronically submitted via EFS-Web to the United States Patent and Trademark Office as text filed named “20110207_SP10-069_ST25.txt” having a size of 6 kb and created on Feb. 07, 2011. Due to the electronic filing of the Sequence Listing, the electronically submitted Sequence Listing serves as both the paper copy required by 37 CFR §1.821(c) and the CRF required by §1.821(e). The information contained in the Sequence Listing is hereby incorporated herein by reference.

### BACKGROUND

Microcarriers have been employed in cell culture for the purpose of providing high yields of attachment-dependent cells. Microcarriers are typically stirred or agitated in cell culture media and provide a very large attachment and growth surface area to volume ratio relative to more traditional culture equipment.

Most currently available microcarriers provide for non-specific attachment of cells to the carriers for cell growth. While useful, such microcarriers do not allow for biospecific cell adhesion and thus do not readily allow for tailoring of characteristics of the cultured cells. For example, due to non-specific interactions it may be difficult to maintain cells, such as stem cells, in a particular state of differentiation or to direct cells to differentiate in a particular manner.

Some currently available microcarriers provide for bio-specific adhesion, but employ animal derived coating such as collagen or gelatin. Such animal derived coatings can expose the cells to potentially harmful viruses or other infectious agents which could be transferred to patients if the cells are used for a therapeutic purpose. In addition, such viruses or other infectious agents may compromise general culture and maintenance of the cultured cells. Further, such biological products tend to be vulnerable to batch variation and limited shelf-life.

Some synthetic, chemically-defined surfaces have been shown to be effective in culturing cells, such as embryonic stem cells, in chemically defined media. However, the ability of such surfaces to support 3D culture on microcarriers has not yet been reported and methods for applying such surfaces to microcarriers have not yet been described.

### BRIEF SUMMARY

Among other things, the present disclosure describes synthetic, chemically-defined microcarriers useful in culturing cells. The microcarriers, in various embodiments, are formed by a one-pot in situ polymerization reaction in which an initiator-conjugated microcarrier base is contacted with one or more monomers and the initiator is activated to initiate polymerization and to graft the polymer from the base via the initiator or a remnant thereof. At least one of the monomers is conjugated to a polypeptide so that incorporation of the monomer into the forming polymer conjugates the polypeptide to the polymeric coating as it is formed in situ. The polymeric coating of the resulting microcarriers should not readily delaminate due to being conjugated to the microcarrier base.

One or more of the various embodiments presented herein provide one or more advantages over prior articles and systems for culturing cells. For example, synthetic microcarriers described herein have been shown to support cell adhesion without the need of animal derived biocoating which limits the risk of pathogen contamination. This is especially relevant when cells are dedicated to cell therapies. Further, large scale culture of cells, including human embryonic stem cells (hESCs) and human mesenchymal stem cells (hMSCs), is possible with microcarriers as described herein. Such microcarriers may also be advantageously used for culturing cells other than stem cells when animal derived products such as collagen, gelatin, fibronectin, etc. are undesired or prohibited. The methods described herein allow for the preparation of microcarriers having a wide range of properties such as stiffness, swellability, density and surface chemistries. Further, embodiments of processes for forming the microcarriers described herein can result in improved efficiency (e.g., one-pot synthesis) and lower costs (due to increased efficiency of peptide conjugation relative to other processes). These and other advantages will be readily understood from the following detailed descriptions when read in conjunction with the accompanying drawings.

### BRIEF DESCRIPTION OF THE DRAWINGS

FIG. 1 is a schematic drawing showing a one pot in situ polymerization coating and polypeptide grafting from the surface of a microcarrier.

FIG. 2 is a schematic drawing of a cross-section of an embodiment of a coated microcarrier having a conjugated polypeptide.

FIG. 3 is a flow diagram of an embodiment of a method of forming a coated microsphere.

FIGS. 4-5 are examples of reaction schemes that may be employed to graft a polymeriziation initiator to a surface of a microcarrier base.

FIG. 6 is a reaction scheme for one pot grafting of a synthetic polymer to initiator-conjugated microcarrier base and conjugating a polypeptide.

FIG. 7 is a bar graph showing estimated polypeptide density grafted to various microcarriers.

FIGS. 8A-B are brightfield images of human mesenchymal stem cells (hMSC) on control microcarriers having no bound vitroncectin (VN) polypeptide (A) and microcarriers having bound VN (B) after four days of culture.

FIGS. 9A-B are brightfield images of hMSC on microcarriers having bound VN at one day (A) and four days (B) of culture.

FIGS. 10A-F are brightfield images of hMSC on microcarriers having bound VN on newly added beads (A, B, C) and old beads (D, E, F) cultured in media with serum (A, D) and medium without serum (B, C, E, F) after two days of culture.

FIG. 11A-D are brightfield images of hMSC on microcarriers having bound VN (A-C) and control microcarriers without bound VN (D) in a serum-containing medium (A, D) and serum-free media (B, C) after three days of culture following the addition of newly added beads.

FIGS. 12A-D are brightfield (left) and fluorescent (right) images of hMSC immunostained with mouse anti-H-CAM (CD44) after three days of expansion on microcarriers having bound VN (A-C) and control microcarriers without bound VN (D) in a serum-containing medium (A, D) and serum-free media (B, C).

The schematic drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

Polypeptide sequences are referred to herein by their one letter amino acid codes and by their three letter amino acid codes. These codes may be used interchangeably.

As used herein, “monomer” means a compound capable of polymerizing with another monomer, (regardless of whether the “monomer” is of the same or different compound than the other monomer), which compound has a molecular weight of less that about 1000 Dalton. In many cases, monomers will have a molecular weight of less than about 400 Dalton.

As used herein, “microcarrier” means a small discrete particle for use in culturing cells and to which cells may attach. Microcarriers may be in any suitable shape, such as rods, spheres, and the like. In many embodiments, a microcarrier includes a microcarrier base that is coated to provide a surface suitable for cell culture. A polypeptide may be bonded, grafted or otherwise attached to the surface coating.

As used herein “peptide” and “polypeptide” mean a sequence of amino acids that may be chemically synthesized or may be recombinantly derived, but that are not isolated as entire proteins from animal sources. For the purposes of this disclosure, peptides and polypeptides are not whole proteins. Peptides and polypeptides may include amino acid sequences that are fragments of proteins. For example peptides and polypeptides may include sequences known as cell adhesion sequences such as RGD. Polypeptides may be of any suitable length, such as between three and 30 amino acids in length. Polypeptides may be acetylated (e.g. Ac-LysGlyGly) or amidated (e.g.SerLysSer-NH₂) to protect them from being broken down by, for example, exopeptidases. It will be understood that these modifications are contemplated when a sequence is disclosed.

As used herein, “equilibrium water content” refers to water-absorbing characteristic of a polymeric material and is defined and measured by equilibrium water content (EWC) as shown by Formula 1:

Formula 1: EWC (%)=[(Wgel−Wdry)/(Wgel)]*100.

As used herein, a “remnant” of a polymerization initiator means a portion of the initiator that results from activation of the initiator to produce free radicals. For example, a polymerization initiator may form a free radical-containing remnant following thermal, photolytic or catalytic activation, which result in inter- or intra-molecular bond dissociation, hydrogen abstraction or other known initiator mechanisms.

As used herein, “have”, “having”, “include”, “including”, “comprise”, “comprising” or the like are used in their open ended sense, and generally mean “including, but not limited to”. It will be understood that “consisting essentially of”, “consisting of”, and the like are subsumed in “comprising” and the like. Accordingly, a microcarrier comprising a microcarrier base and a coating includes a microcarrier consisting essentially of, or consisting of, a microcarrier base and a coating.

The present disclosure describes, inter alia, synthetic microcarriers for culturing cells. In various embodiments, the microcarriers are configured to support proliferation and maintenance of undifferentiated stem cells in chemically defined media. The microcarriers described herein include a microcarrier base, a polymeric coating conjugated to the base, and a polypeptide conjugated to the polymer coating.

In various embodiments, the microcarriers are formed by a one-pot in situ polymerization reaction in which an initiator-conjugated microcarrier base is contacted with one or more monomers and the initiator is activated to initiate polymerization and to graft the polymer from the base via the initiator or a remnant thereof. At least one of the monomers is conjugated to a polypeptide so that incorporation of the monomer into the forming polymer conjugates the polypeptide to the polymeric coating as it is formed in situ.

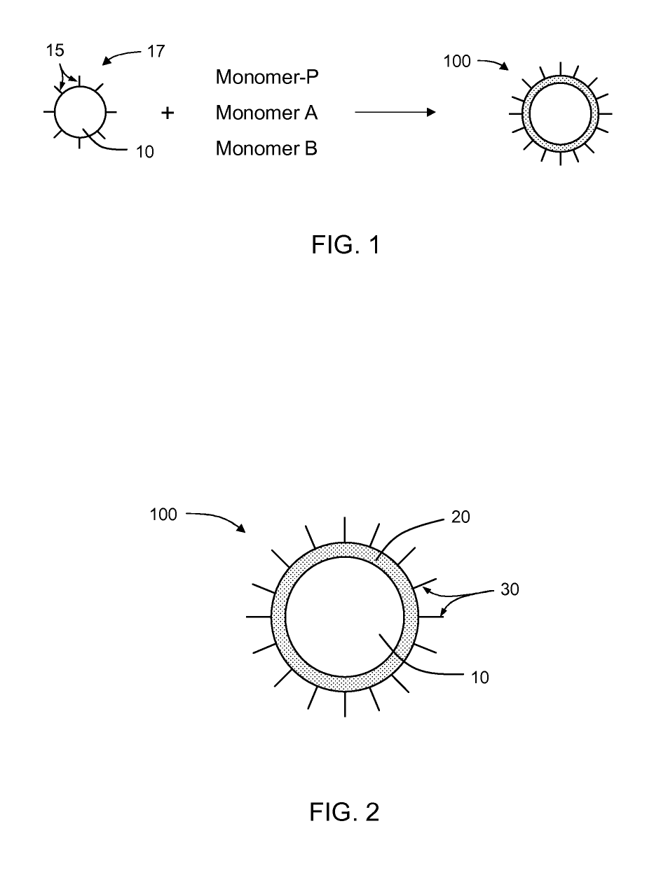

For example and with reference to FIG. 1, a reaction scheme showing the general process is described. An initiator conjugated microcarrier base 17 includes, or consists essentially of, a microcarrier base 10 and a polymerization initiator 15 conjugated to the surface of the base 10. The initiator conjugated microcarrier base 17 brought into contact with one or more monomers (e.g., monomer A, monomer B, and monomer-P, as depicted), at least one of which is conjugated to a polypeptide (monomer-P). The initiator is activated and a microcarrier 100 is formed.

1. Microcarrier

Referring now to FIG. 2, a microcarrier 100, regardless of how formed, includes a base 10, a coating 20 and a conjugated polypeptide 30. The polypeptide 30 alone or the coating 20 and polypeptide 30 together provide a surface to which cells can attach for the purposes of cell culture. In some circumstances, it may be desirable for the cells to specifically attach to the polypeptide 30 with little or no non-specific binding to the coating 20. In various embodiments, the coating layer 20 and polypeptide 30 are deposited on or formed on a surface of an intermediate layer that is associated with the base material 10 via covalent or noncovalent interactions, either directly or via one or more additional intermediate layers (not shown). In such cases, the intermediate is considered, for the purposes of this disclosure, to be a part of the microcarrier base 10.

Microcarriers can have any suitable density. However, it is preferred that microcarriers have a density slightly greater than the cell culture medium in which they are to be suspended to facilitate separation of the microcarriers from the surrounding medium. In various embodiments, the microcarriers have a density of about 1.01 to 1.10 grams per cubic centimeter. Microcarriers having such a density should be readily maintained in suspension in cell culture medium with gentle stirring.

It is also preferred that the size variation of the microcarriers is small to ensure that most, if not all, of the microcarriers can be suspended with gentle stirring. By way of example, the geometric size distribution of the microcarriers may be between about 1 and 1.4. Microcarriers may be of any suitable size. For example, microcarriers may have a diametric dimension of between about 20 microns and 1000 microns. Spherical microcarriers having such diameters can support the attachment of several hundred to thousands of cells per microcarrier. The size of the microcarrier bases, and thus the overall microcarrier, can be readily controlled via known techniques. By way of example, microcarrier bases formed via water-in-oil copolymerization techniques can be easily tuned by varying the stirring speed or the type of emulsifier used. For example, higher stirring speeds tend to result in smaller particle size. In addition, it is believed that the use of polymeric emulsifiers, such as ethylcellulose, enables larger particles relative to lower molecular weight emulsifiers. Accordingly, one can readily modify stirring speed or agitation intensity and emulsifier to obtain microcarrier bases of a desired particle size.

Microcarriers can be porous or non-porous. As used herein, “non-porous” means having no pores or pores of an average size smaller than a cell with which the microcarrier is cultured, e.g., less than about 0.5-1 micrometers. Non-porous microspheres are desired when the microcarriers are not degradable, because cells that enter pores of macroporous microcarriers are difficult to remove. However, if the microcarriers are degradable, e.g. if they include an enzymatically or otherwise degradable cross-linker, it may be desirable for the microcarriers to be macroporous.

2. Microcarrier Base

Any suitable microcarrier base may be used. In various embodiments the microcarrier base is formed from glass, ceramic, metal or polymeric material. Examples of polymeric materials that can be used to create microcarriers include thermoplastic or thermoset polymers, hydrogels, and biodegradable polymers. For example the microcarrier may be formed from polystyrenes, acrylates such as polymethylmethacryate, acrylamides, agarose, polysaccharides such as dextrans, gelatins, latexes, and the like, or combinations thereof. Examples of biodegradable polymers that may be used to form the microcarrier base include poly-lactic acid, poly-caprolactone, poly-buteryrate-co-valerate, and the like, or combinations thereof. The microcarrier base may have special characteristics such as being magnetic to ease separation from bulk media. In some embodiments, the microcarriers are microspheres, many of which are commercially available. Microspheres can be produced by any suitable method and are typically produced by suspension polymerization of a “water-in-oil”-type emulsion.

3. Coating

A microcarrier base may be coated with polymer from any suitable class of biocompatible polymers such as poly(meth)acrylates, polyamides, polyphosphazenes, polypropylfumarates, synthetic poly(amino acids), polyethers, polyacetals, polycyanoacrylates, poly(meth)acrylamides, polyurethanes, polycarbonates, polyanhydrides, poly(ortho esters), polyhydroxyacids, polyesters, ethylene-vinyl acetate polymers, cellulose acetates, polystyrenes, poly(vinyl chloride), poly(vinyl fluoride), poly(vinyl imidazole), poly(vinyl alcohol), chlorosulphonated polyolefins, and combinations thereof.

“Coating”, “layer”, “surface”, “material”, and the like are used interchangeably herein, in the context of a polymer disposed on a microcarrier base. Preferably, the coating is a synthetic polymer coating free from animal-derived components, as animal derived components occasionally may contain viruses or other infectious agents or may provide a high level of batch-to-batch variability. In various embodiments, the coating is a hydrogel coating or a swellable (meth)acrylate coating, e.g., as described in U.S. patent application Ser. No. 12/362,924, filed on Jan. 30, 2009, entitled SYNTHETIC SURFACES FOR CULTURING CELLS IN CHEMICALLY DEFINED MEDIA, and published on Jul. 30, 2009 as US 2009/0191627; and U.S. patent application Ser. No. 12/362,974, filed on Jan. 30, 2009, entitled SWELLABLE (METH)ACRYLATE SURFACES FOR CULTURING CELLS IN CHEMICALLY DEFINED MEDIA, and published on Jul. 30, 2009 as US 2009/0191632, which applications are hereby incorporated herein by reference in their respective entireties to the extent that they do not conflict with the disclosure presented herein.

As used herein, “swellable (meth)acrylate” or “SA” means a polymer matrix made from at least one ethylenically unsaturated monomer (acrylate or methacrylate monomers) having at least some degree of cross linking, and also having water absorbing or water swelling characteristics. “SAP”, as used herein, means as SA conjugated to a polypeptide or protein. In embodiments, the term “swellable (meth)acrylate” represents a range of cross-linked acrylate or methacrylate materials which absorb water, swell in water, and do not dissolve in water.

In various embodiments, the SA coating comprises, consists essentially of, or consists of, reaction products of one or more hydrophilic (meth)acrylate monomer, one or more di- or higher-functional (meth)acrylate monomer (“cross-linking” (meth)acrylate monomer), and one or more monomers conjugated to a polypeptide. Any suitable hydrophilic (meth)acrylate monomer may be employed. Examples of suitable hydrophilic (meth)acryate monomers include 2-hydroxyethyl methacrylate, di(ethylene glycol)ethyl ether methacrylate, ethylene glycol methyl ether methacrylate, and the like. In various embodiments, hydrophilic monomers other than (meth)acrylates may be used to form the SA coating. These other hydrophilic monomers may be included in addition to, or in place of, hydrophilic (meth)acrylate monomers. Such other hydrophilic monomers should be capable of undergoing polymerizing with (meth)acrylate monomers in the mixture used to form the swellable (meth)acrylate layer. Examples of other hydrophilic monomers that may be employed to form the SA coating include 1-vinyl-2-pyrrolidone, acrylamide, 3-sulfopropyldimethyl-3-methylacrylamideopropyl-ammonium, and the like. Regardless of whether a (meth)acrylate monomer or other monomer is employed, a hydrophilic monomer, in various embodiments, has a solubility in water of 1 gram or more of monomer in 100 grams of water. Any suitable di- or higher-functional (meth)acrylate monomer, such as tetra(ethylene glycol) dimethacrylate or tetra(ethylene glycol) diacrylate, may be employed as a cross-linking monomer. Any suitable monomer conjugated to a polypeptide may be employed. In various embodiments the monomer conjugated to the polypeptide is a (meth)acrylate monomer, a (meth)acrylamide monomer, or the like.

A polymer coating layer may have any desirable thickness. In various embodiments, the average thickness of the coating layer is less than about 100 micrometers. For example, the average thickness may be less than about 50 micrometers, less than about 20 micrometers, less than about 10 micrometers, less than about 5 micrometers, less than about 2 micrometers, less than about 1 micrometer, less than about 0.5 micrometers, between about 50 nm and about 300 nm, or about 0.1 micrometers. It will be understood that the coating thickness will depend on several variables, including the grafting technique employed, the reaction conditions, the reaction time, and the technique used to measure the coating thickness. For example, coating thickness measured by SEM in the dry state may be different than if measured in its hydrated state using a technique such as confocal microscopy in buffered solutions.

4. Coating of Microcarrier Base With Polymer

Monomers are brought in contact with the surface of a microcarrier base and polymerized in situ. In such embodiments, the base will be referred to herein as the “substrate” on which the polymer is deposited or formed. Polymerization may be done in solution phase or in bulk phase. The substrate is suspended in the monomer mixture and polymerization is initiated at the surface of the substrate. As monomers may be viscous, it may be desirable to dilute the monomers in a suitable solvent to reduce viscosity prior to suspending the microcarrier base substrate. Reducing viscosity may allow for thinner and more uniform layers of the coating material to be formed. Preferably the solvent is compatible with the microcarrier base material and the monomers. It may be desirable to select a solvent that is nontoxic to the cells to be cultured and that does not interfere with the polymerization reaction. Alternatively, or in addition, selection of a solvent that can be substantially completely removed or removed to an extent that it is non-toxic or no longer interferes with polymerization may be desirable. In such circumstances, it may be desirable that the solvent be readily removable without harsh conditions, such as vacuum or extreme heat. Volatile solvents are examples of such readily removable solvents.

Some solvents that may be suitable in various situations for coating articles as described herein include methanol, ethanol, acetone, butanone, acetonitrile, 2-butanol, isopropanol, acetyl acetate, ethyl acetate, dimethylformamide (DMF), dimethylsulfoxide (DMSO), water or combinations thereof.

The monomers may be diluted with solvent by any suitable amount to achieve the desired viscosity and monomer concentration. For example, the monomer compositions may contain between about 0.1% to about 99% monomer. By way of example, the monomer may be diluted with an ethanol or other solvent to provide a composition having between about 0.1% and about 50% monomer, or from about 0.1% to about 10% monomer by volume, or from about 0.1% to about 1% monomer by volume. The monomers may be diluted with solvent so that the coating layer achieves a desired thickness. The layer thickness may also be controlled by polymerization reaction time, monomer to initiator concentration ratio, or the like.

In addition to the monomers that form the coating layer, a composition forming the layer may include one or more additional compounds such as surfactants, wetting agents, polymerization initiators, catalysts or activators.

Whether polymerized in bulk phase (substantially solvent free) or solvent phase, the monomers are polymerized via an appropriate initiation mechanism. Many of such mechanisms are known in the art. For example, temperature may be increased to activate a thermal initiator, photoinitiators may be activated by exposure to appropriate wavelength of light, redox systems may be activated by oxidation reduction chemical initiator pairing, or the like. Polymerization may be carried out under inert gas protection, such as nitrogen protection, to prevent oxygen inhibition.

Any suitable polymerization initiator that can be immobilized may be employed. One of skill in the art will readily be able to select a suitable initiator, e.g. a radical initiator or a cationic initiator, suitable for use with the monomers. Examples of polymerization initiators include organic peroxides, azo compounds, quinones, nitroso compounds, acyl halides, hydrazones, mercapto compounds, pyrylium compounds, imidazoles, chlorotriazines, benzoin, benzoin alkyl ethers, diketones, phenones, diethyl dithiocarbamates, bromo or hydroxy acids or acid halides, or mixtures thereof. Preferably, the initiator is capable of providing surface initiated grafting of the forming polymer (i.e., grafting from the surface) and minimizes grafting in solution. Grafting from the surface is desirable when the forming polymer is cross-linked (i.e., formed from one or more di- or higher functional monomer). Non-limiting examples of monomers capable of grafting from the surface with minimal or no transfer of radicals away from the surface include 4,4′-Azobis-(4-cyanopentanoic acid) (ACBA), 4-(3-hydridodiethylsilyl)propyloxybenzophone, (3 -(2 -bro mo is obutyryl)propyl) diethylhydridosilane, and 2-bromo-isobutyryl bromide. For some initiators, such as ACBA, which splits to form two radicals, the initiator is preferably anchored to the surface via two anchoring groups such that each radical containing moiety remains bound to the surface and does not migrate away from the surface.

The initiator may be conjugated or immobilized (i.e., covalently bound) to the microcarrier base via any suitable method. To facilitate the conjugation of an initiator to the surface of a microcarrier base, the microcarrier base may include a functional group suitable for reaction with the initiator. The microcarrier base may include any suitable functional group, and the suitability of the functional group may depend on the initiator used. For example, if either one of the functional group of the microcarrier base or the polymerization initiator has an available carboxylic acid group, the hydroxyl group of the carboxylic acid may be replaced by a suitable nucleophile, such as a nitrogen of an amine (via an amidation reaction) or an oxygen of an alcohol (via an esterification reaction). By way of further example, the microcarrier may be glass (or contain an available silanol group) and the polymerization initiator may have an available silane coupling group (or hydrolysable group such as alkoxy, acyloxy, or halogen). Not to be bound by theory, it is believed that the hydrolysable group of the polymerization initiator first hydrolyzes, condenses (loss of water) into silanol-oligomer and then hydrogen bonds to the OH groups of the glass. Heat is then introduced to promote condensation resulting in a covalent linkage formed with the initiator and the glass microcarrier. These aforementioned reactions may all occur simultaneously after the initial hydrolysis of the hydrolysable groups.

Factors such as initial concentration of surface hydroxyls, type of surface hydroxyls, stability of the bond formed and dimensions/features of the substrate may influence the effectiveness of the initiator coupling. It is often desirable to have the maximum number of accessible reactive sites on the glass microcarrier to maximize initiator coupling. Acid or base etching (e.g., 1M sodium hydroxide, ammonia, hydrochloric acid), UV-ozone, or plasma treatment may be included as a step to pretreat the glass microcarrier to clean and/or expose more reactive silanol groups which may interact with the silane-initiator. Other hydroxyl-containing substrates such as silica, quartz, alimunium, alumino-silicates, copper inorganic oxides, etc. may be used as an alternative to glass.

In some embodiments, the initiator is selected and immobilized in a way so that polymerization takes place at the surface of the microcarrier base with minimal polymerization in solution, which may be desirable in situations where insoluble crosslinked polymers, which can become difficult to remove from the bulk polymerization mixture, are grafted. Surface inititators include reversible addition-fragmentation chain transfer (RAFT), atom transfer radical polymerization (ATRP), or other surface initiators. Suitable activators, as readily identifiable in the art, may be employed to facilitate such reactions.

Examples of suitable reactions may include, amide bond formation using EDC/NHS activation, HATU/DIEA, EEDQ and other amide bond forming reactions. Initiators containing isothiocyanate, isocyanate, acyl halde, aldehyde, epoxy, anhydride, or other amine reactive groups may be immobilized onto an amine containing microcarrier base support. Initiators containing, maleimides, thiols, and the like may be immobilized onto thiol microcarrier base supports by Michael addition or disulfide forming reactions. Initiators containing hydroxyl or amine groups can be immobilized onto epoxide or oxiraine microcarrier base supports by nucleophilic ring opening mechanisms. Cycloaddition reactions such as click chemistry, Diels-Alder reactions may be employed as immobilization methods. Affinity reactions such as biotin/Streptavidin, or protein A/immunoglobulin G interactions may be used for initiator immobilization. The support or the initiator may contain the appropriate function group to facilitate immobilization. For example, as described herein, a carboxylic acid containing initiator may be immobilized onto an amine containing microcarrier base support using EEDQ activation of the COOH-containing initiator. The reverse scenario, i.e., an amine containing initiator being immobilized to a COOH microcarrier base support, can also be envisioned. The other surface conjugation techniques described in the literature may also be applied for initiator immobilization. Such techniques have been thoroughly reviewed in the literature (Hermanson, G. T. Bioconjugate Techniques. Second Edition; Academic Press; Elsevier Inc. 2008).

By way of example, polymerization initiators having available carboxylic acid groups include 4,4′-azobis(4-cyanovaleric acid) (ABCA), and 4-benzoyl benzoic acid. Non-limiting examples of polymerization initiators having available hydroxyl groups include 2,2′-azobis[2-methyl-N-(2-hydroxyethyl)propionamide] and 2,2′-azobis{2-methyl-N-[1,1-bis(hydroxymethyl)-2-hydroxyethl]propionamide} available from Wako Specialty chemicals under the trade name VA-086 or VA-080 respectively. Examples of photolyzable or ATRP initiators containing silane groups include 4-(3-hydridodiethylsilyl)propyloxybenzophone or (3-(2-bromoisobutyryl)propyl) diethylhydridosilane, respectively. An example of amine reactive ATRP initiator is 2-bromo-isobutyryl bromide available from Sigma. An example of a nucleophilic photo chain transfer initiator is diethyldithiocarbamate sodium salt available from GangFu Fine chemicals.

As mentioned above, a photoinitiator containing a silane coupling group may readily conjugated to glass (or other hydroxide-containing substrate; e.g. most inorganics). For example, such an initiator may be conjugated to a glass microcarrrier or a microcarrer having available hydroxyl groups by contacting the initiator with the microcarrier, followed by a heat promoted condensation step; e.g. as discussed above.

Any suitable initiator may be formed into a silyl ether. In various embodiments the silyl ether initiator is of the following formula:

In various embodiments R₁, R₂, and R₃ are each independently substituted or unsubstituted C1-C3 alkyl, alkoxy, or hydrogen; and X is C1-C6 straight or branched chain substituted or unsubstituted alkyl and may be present or absent. By way of example, the silyl ether initiator is an alkoxy-substituted silyl benzophenone, such as those described in U.S. Pat. Nos. 4,495,360 and 4,278,804. One suitable alkoxy-substituted silyl benzophenone is 2-hydroxy-4(3-triethxysilylpropoxy)-dephenylketone (HDPK-Si).

In general, the amount of immobilized initiator will depend on the functional group loading of the microcarrier base support. Typically for crosslinked polystyrene, functionalities from 0.1 to 2 mmol/g of bead are available for various functional groups such as hydroxyl, amino and carboxylic acid. In some embodiments, the initiator is immobilized in a way so that polymerization takes place at the surface of the microcarrier base with minimal polymerization in solution, which may be desirable in situations where insoluble crosslinked polymers are grafted. In various embodiments, the immobilization level of the initiator is less than about 100% of the initial reactive functional group loading. For example, initiator level may be less than about 75% of the initial reactive functional group loading, less than about 50% of the initial reactive functional group loading, less than about 25% of the initial reactive functional group loading, less than 10% of the initial reactive functional group loading, less than about 5% of the initial reactive functional group loading, or about 1% of the functional group loading. By way of example, the polymerization initiator, 4,4′-azobis(4-cyanovaleric acid) (ABCA), is immobilized so that all carboxylic acid groups are tied to the surface of the microcarrier base support. When the azo initiator fragments, both free radical groups remain tied to the surface with minimal radicals in the bulk solution. Similarly, the conjugation of the benzophenone from 2-hydroxy-4(3-triethxysilylpropoxy)-dephenylketone (HDPK-Si) to the surface of a glass microcarrier leaves the initiator bound to the surface by intermolecular hydrogen abstraction. By immobilizing both of the free radical groups to the base, bulk polymerization in solution can be limited.

A photosensitizer may also be included in a suitable initiator system. Representative photosensitizers have carbonyl groups or tertiary amino groups or mixtures thereof. Photosensitizers having a carbonyl groups include benzophenone, acetophenone, benzil, benzaldehyde, o-chlorobenzaldehyde, xanthone, thioxanthone, 9,10-anthraquinone, and other aromatic ketones. Photosensitizers having tertiary amines include methyldiethanolamine, ethyldiethanolamine, triethanolamine, phenylmethylethanolamine, and dimethylaminoethylbenzoate. Commercially available photosensitizers include QUANTICURE ITX, QUANTICURE QTX, QUANTICURE PTX, QUANTICURE EPD from Biddle Sawyer Corp. However, if a photosensitizer is used, it preferably minimizes polymerization in solution.

The cured coating layer may be washed with solvent one or more times to remove impurities such as unreacted monomers or low molecular weight polymer species. In various embodiments, the layer is washed with ethanol or an ethanol/water solution, e.g. 50% ethanol, 70% ethanol, greater than 90% ethanol, greater than 95% ethanol or greater than about 99% ethanol. The size and shape of the base microcarrier support allows facile and thorough washing of the coated microcarrier substrate. Any suitable filter apparatus may be incorporated to remove the washing solvent. Examples of filter systems are peptide synthesis vessels equipped with a vacuum filter or a soxhlet apparatus for higher temperature washings.

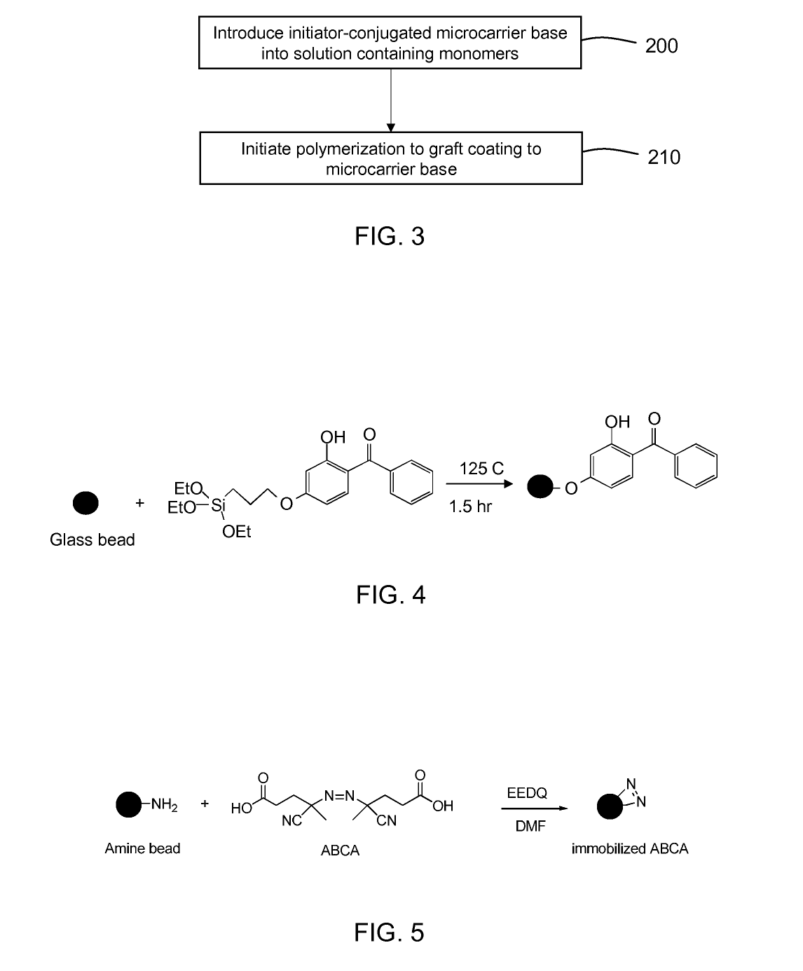

Referring now to FIG. 3, the polymer layer may be grafted (e.g., covalently bound) to the microcarrier base as it is formed in situ while in contact with the microcarrier base. As shown in FIG. 3, in various embodiments, a method for grafting a coating layer to a microcarrier includes (i) introducing an initiator conjugated microcarrier base into a solution containing monomers (200), and (ii) initiating polymerization to graft the coating to the microcarrier base in situ while in contact with the base (210). At least one of the monomers includes a conjugated peptide so that the polymerization results in the polypeptide being bound to the microcarrier base via the polymeric coating.

By employing the methods outlined above (e.g., with regard to FIGS. 1 and 3), a coated microcarrier is produced, where the coating is grafted to the microcarrier base via a polymerization initiator. Such grafting of the coating may provide for improved integrity of the coating and reduced delamination during cell culture. It is further noted that the process outlined herein produces much more efficient grafting of polypeptide to the microcarrier than alternative methods, such as those described in U.S. Provisional Application Ser. No. 61/229,114, filed on Jul. 28, 2009, having attorney docket number of SP09-221P. In that provisional application, a carboxylic acid containing monomer was used to form the coating of the microcarrier, and EDC/NHS chemistry was employed to conjugate the polypeptide to the coating after the coating was formed. The present process has been found to result in a 6 to 10 fold increase in peptide density while using two-thirds less polypeptide. Thus, the presently described process enables a lower cost method to prepare peptide conjugated microcarriers.

5. Initiator Conjugated Microcarrier Base

Any suitable initiator conjugated base may be employed in accordance with the teachings presented herein. Polymerization initiators may be conjugated to microcarrier bases in any suitable fashion. In many embodiments, a polymerization imitator is conjugated to a pendant functional group of a microcarrier base.

Many suitable functionalized microcarrier substrates are available from commercial sources. For example, COOH, SH, NH₂, and CHO functionalized polystyrene resins and microspheres are available from Rapp Polymere GMBH; amino, carboxylate, carboxy-sulfate, hydroxylate, and sulfate functionalized polystyrene beads are available from Polysciences, Inc.; and amine functionalized glass beads available from Polysciences, Inc. Carboxylate functionalzed dextran beads are available from GE Healthcare, Hyclone, and Sigma-Aldrich. Azlactone functionalized beads are available from Pierce. Unfunctionalized magnetic beads are available from Merck.

Of course, functional groups may readily be added to microcarriers via techniques known in the art. For example, glass carriers may be readily functionalized with an appropriate organosilane. It may be desirable to treat or etch the surface of the glass carrier prior to functionalization to increase surface area. Functionalized epoxy resins may be employed to functionalize glass or other suitable microcarriers. Polystyrene or other suitable microcarriers can also be readily functionalized using known techniques. For example, a microcarrier base may be prepared by polymerization of monomers such as chloromethylstyrene or 4-t-BOC-hydroxystyrene. Other suitable monomers are styrene, a-methylstyrene, or other substituted styrene or vinyl aromatic monomers that, after polymerization, can be chloromethylated to produce a reactive microcarrier intermediate that can be subsequently converted to a functionalized microcarrier. Of course, monomers that do not bear reactive groups (including the crosslinking agent) can be incorporated into the microcarrier. Chemical modification of the reactive microcarrier intermediate may be carried out by a variety of conventional methods.

With reference now to FIG. 4, an example of one suitable reaction scheme for immobilizing a polymerization initiator on a microcarrier is shown. In the scheme shown in FIG. 4, a siloxane modified photoinitiator, HDPK-Si, having silyl ether functionality is conjugated to a glass microcarrier (glass bead) using under heat, leaving the bead with hydroxyl diphenylketone (HDPK) conjugated via an ether linkage. Such initiator-conjugated microcarriers may be placed in solution with appropriate monomers in an appropriate solvent (e.g., methanol) and subjected to UV radiation to initiate polymerization to produce a coated microcarrier as described above.

With reference now to FIG. 5, another example of one suitable reaction scheme for immobilizing a polymerization initiator on a microcarrier is shown. In the scheme shown in FIG. 5, a thermal initiator, ABCA, having a carboxylic acid functionality (in this case two carboxylic acid groups) is conjugated to an amine-functionalized microcarrier (amine bead) using 2-ethoxy-1-ethoxycarbonyl-1,2-dihydroquinoline (EEDQ) activation. Such initiator-conjugated microcarriers may be placed in solution with appropriate monomers in an appropriate solvent (e.g., methanol) and heated to initiate polymerization to produce a coated microcarrier as described above.

It will be understood that the reaction scheme shown in FIGS. 4-5 are only some examples of a reaction schemes that may be employed to graft a coating to a microcarrier base, and that any suitable reaction scheme may be employed, depending on the functional groups available on the microcarrier base and the initiator. For example, a drying step may be employed between steps, such as after the polymerization step and before grafting peptides. However, this and other drying steps may not be necessary. If drying steps are not employed, the surface may be washed using the buffer or solution called for in the next manufacturing step. Further, it will be understood, that while the schemes in FIGS. 4-5 show the use of a photoinitiator or a thermal initiators, RAFT (reversible addition fragment chain transfer) initiator, ATRP (atom transfer radical polymerization) initiators, or other surface initiators may be employed to graft the polymer.

While described with regard to microcarriers, it will be understood that the methods described herein can be used to graft a polymeric coating in situ to a surface of any cell culture article. For example, a polymer layer may be grafted to one or more surfaces of a multi-well plate, a jar, a petri dish, a flask, a multi-layered flask, a beaker, a plate, a roller bottle, a slide, such as a chambered or multi-chambered slide, a tube, a coverslip, a bag, a membrane, a hollow fiber, a cup, a spinner bottle, a perfusion chamber, a bioreactor, or a fermentor. The substrate or base material of such cell culture articles may be formed from any suitable material such as a metallic surface, a ceramic substance, a glass, a plastic, a polymer or co-polymer, any combinations thereof, or a coating of one material on another. For example, the substrate or base materials may include glass materials such as soda-lime glass, pyrex glass, vycor glass, quartz glass; silicon; plastics or polymers, including dendritic polymers, such as poly(vinyl chloride), poly(vinyl alcohol), poly(methyl methacrylate), poly(vinyl acetate-co-maleic anhydride), poly(dimethylsiloxane) monomethacrylate, cyclic olefin polymers, fluorocarbon polymers, polystyrenes, polypropylene, polyethyleneimine; copolymers such as poly(vinyl acetate-co-maleic anhydride), poly(styrene-co-maleic anhydride), poly(ethylene-co-acrylic acid) or derivatives of these or the like. Such material may be readily functionalized as described herein or as known in the art.

6. Polypeptide Conjugated to Monomer

Any suitable polypeptide may be conjugated to a monomer for incorporation into the polymeric coating. In various embodiments, polypeptides or proteins are synthesized or obtained through recombinant techniques, making them synthetic, non-animal-derived materials. A polymerizable group may be incorporated into the polypeptide by techniques known in the art. For example, any free amine or hydroxyl of the polypeptide (via lysine, serine, etc.) may be (meth)acrylated using (meth)acryloyl chloride or (meth)acrylic anhydride.

In numerous embodiments, the polypeptide, or a portion thereof, has cell adhesive activity; i.e., when the polypeptide is conjugated to the coated microcarrier, the polypeptide allows a cell to adhere to the surface of the peptide-containing coated microcarrier. By way of example, the polypeptide may include an amino sequence, or a cell adhesive portion thereof, recognized by proteins from the integrin family or leading to an interaction with cellular molecules able to sustain cell adhesion. For example, the polypeptide may include an amino acid sequence derived from collagen, keratin, gelatin, fibronectin, vitronectin, laminin, bone sialoprotein (BSP), or the like, or portions thereof In various embodiments, polypeptide includes an amino acid sequence of ArgGlyAsp (RGD).

Microcarriers as described herein provide a synthetic surface to which any suitable adhesion polypeptide or combinations of polypeptides may be conjugated, providing an alternative to biological substrates or serum that have unknown components. In current cell culture practice, it is known that some cell types require the presence of a biological polypeptide or combination of peptides on the culture surface for the cells to adhere to the surface and be sustainably cultured. For example, HepG2/C3A hepatocyte cells can attach to plastic culture ware in the presence of serum. It is also known that serum can provide polypeptides that can adhere to plastic culture ware to provide a surface to which certain cells can attach. However, biologically-derived substrates and serum contain unknown components. For cells where the particular component or combination of components (peptides) of serum or biologically-derived substrates that cause cell attachment are known, those known polypeptides can be synthesized and applied to a microcarrier as described herein to allow the cells to be cultured on a synthetic surface having no or very few components of unknown origin or composition.

For any of the polypeptides discussed herein, it will be understood that a conservative amino acid may be substituted for a specifically identified or known amino acid. A “conservative amino acid”, as used herein, refers to an amino acid that is functionally similar to a second amino acid. Such amino acids may be substituted for each other in a polypeptide with a minimal disturbance to the structure or function of the polypeptide according to well known techniques. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine (R), Lysine (K), Histidine (H); Acidic: Aspartic acid (D), Glutamic acid (E), Asparagine (N), Glutamine (Q).

A linker or spacer, such as a repeating poly(ethylene glycol) linker or any other suitable linker, may be used to increase distance from polypeptide to surface of the coated microcarrier. The linker may be of any suitable length. For example, if the linker is a repeating poly(ethylene glycol) linker, the linker may contain between 2 and 10 repeating ethylene glycol units. In some embodiments, the linker is a repeating poly(ethylene glycol) linker having about 4 repeating ethylene glycol units. All, some, or none of the polypeptides may be conjugated to a coated microcarrier via linkers. Other potential linkers that may be employed include polypeptide linkers such as poly(glycine) or poly(β-alanine). Any conjugation techniques may be employed to conjugate a linker to the polypeptide. For example, 2-carboxyethylacrylate, mono-(2-methacryoyloxyl)-ethyl succinate, or an acryl-poly(ethylene glycol) succinimide 3400 spacer may be conjugated to the polypeptide by EDC/NHS chemistry to prepare polypeptide monomers of increasing spacer length. In some embodiments, amino acids themselves can serve as linkers or spacers. For example, additional amino acids may be inserted at the N- or C-terminus of a polypeptide to serve as a linker or spacer. In some embodiments the linker includes polylysine, where the linker includes between 1 and 10 repeating lysine units; e.g. between 1 and 4 repeating lysine units.

A polypeptide may be conjugated to the coated microcarrier at any density, preferably at a density suitable to support culture of undifferentiated stem cells or other cell types. Polypeptides may be conjugated to a microcarrier at a density of between about 1 pmol per mm² and about 50 pmol per mm² of surface of the microcarrier. For example, the polypeptide may be present at a density of greater than 5 pmol/mm², greater than 6 pmol/mm², greater than 7 pmol/mm², greater than 8 pmol/mm², greater than 9 pmol/mm², greater than 10 pmol/mm², greater than 12 pmol/mm², greater than 15 pmol/mm², or greater than 20 pmol/mm² of the surface of the coated microcarrier. In cases where the coating is thick (e.g, <1 micrometer) some polypeptide may be conjugated to the subsurface making it challenging to estimate polypeptide density by surface area unit. In this case the polypeptide density may be conjugated at a density between 0.1 nmol/mg and about 1 mmol/mg assuming the microcarrier bulk density is between 1.01 and 1.10 cm2/g. Standard BCA colorimetric techniques may be used to estimate peptide density. It will be understood that the amount of polypeptide present can vary depending on the composition of the coating of the microcarrier, the size of the microcarrier and the nature of the polyp eptide itself.

A polypeptide may be conjugated to a monomer via any suitable manner. In various embodiments, the polypeptide conjugated monomer has the following structure:

monomer-(inker)ₙ-polypeptide,

where n is 0 or 1. For the purposes of this disclosure a linker may be of any suitable length. By way of example, a repeating PEG linker having between 2 and 10 ethylene glycol units will be considered a single linker (i.e., n=1, rather than n=2-10). Any suitable free-radical polymerizable monomer may be employed, such as a (meth)acrylate monomer, including a (meth)acrylamide monomer, a vinyl monomer, or a melamide monomer. In various embodiments, the polypeptide conjugated monomer may have one of the following structures:

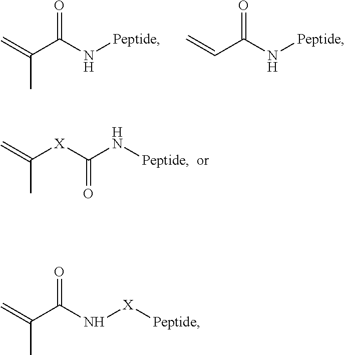

where X may be a linker of any suitable length. In some embodiments, the linker is a hydrophilic linker, such as a PEG lnker, an polyethylene-oxide linker, a polypropylene-oxide linker, or the like. In some embodiments, the linker is a hydrophobic linker, such as an alkyl linker (e.g. a C1-C10 substituted or unsubstituted alkyl), a fatty acid amide linker, or the like. Of course any other suitable linker may be employed. When the linker is a polypeptide linker, the nature of the linker will depend on the amino acids used as the linker. For example, a series of leucines, glycines, or isoleucines can produce a hydrophobic linker or portion thereof. It will be understood that the nature of the linker (e.g., length, hydrophobicity, hydrophilicity, neutral, cationic, etc.) may affect the ability of cells to bind to the active site of the polypeptide (e.g., cell adhesive portion), may affect the non-specific bind of cells to the microcarrier, and the like. For example, the linkers can be used to control the special arrangement and accessibility of the active site of the polypeptide to affect cell binding as desired.

Linkers or spacers, such as poly(ethylene glycol) linkers (e.g., available from Quanta BioDesign, Ltd.) with a terminal amine may be added to the N-terminal amino acid of polypeptide. When adding a linker to the N-terminal amino acid, the linker may be a N-PG-amido-PEGₓ-acid where PG is a protecting group such as the Fmoc group, the BOC group, the CBZ group or any other group amenable to peptide synthesis and X is 2, 4, 6, 8, 12, 24 or any other discrete PEG which may be available.

In some embodiments, the linker is poly(ethylene-oxide) (PEO) having between 1 and 10 repeating ethylene-oxide units, such as 3, 4 or 5 repeating ethylene-oxide units. The PEO linker may be conjugated to the monomer. For example, the PEO linker may be a methylmethacrylate (MMA)-PEO linker/monomer. Such a linker, or other linkers described herein, may be conjugated to the N-terminal amino acid of the polypeptide or the epsilon terminal of a lysine or other suitable amino acid (homolysine, ornithine, etc.).

In some embodiments, the NH depicted above is from an amine of the peptide or a linker conjugated to the polypeptide. The peptide may be reacted with a suitable (meth)acrylic acid, chloride or anhydride to enable nucleophilic attack, e.g. via amide bond formation as shown, to conjugate the peptide to the monomer. By way of example, any native or biomimetic amino acid having functionality that enables nucleophilic addition; e.g. via amide bond formation, may be included in polypeptide for purposes of conjugating to an appropriate (meth)acrylic acid or other suitable monomer. Lysine, homolysine, ornithine, diaminoproprionic acid, and diaminobutanoic acid are examples of amino acids having suitable properties for conjugation to a carboxyl group of a suitable monomer. In addition, the N-terminal alpha amine of a polypeptide may be used to conjugate to a carboxyl group of a monomer, if the N-terminal amine is not capped. In various embodiments, the amino acid of polypeptide that conjugates with a monomer is at the carboxy terminal position or the amino terminal position of the polypeptide. Similarly a carboxylic acid group of a polypeptide may be used to conjugate to an amine group of a monomer.

While the above-discussion is with regard to conjugating a polypeptide to a (meth)acrylate monomer, it will be understood that any monomer having an available carboxylic acid group may be conjugated to a polypeptide in a similar manner. Thus, the peptide conjugated monomer may have a structure of: peptide-NH-C(O)-monomer (shown without linkers). Further, it will be understood that nucleophilic addition is but one way in which a polypeptide may be conjugated to a monomer.

Several vendor offer monomers conjugated to polypeptides for sale. For example, (meth)actylate monomers conjugated to polypetides may be purchased from American Peptide Company, Inc., for example, Ac-Lys(2-carboxyethyl acrylate)-GGPQVTRGDVFTMP-NH2 (product 348849), MAA-Lys(Ac)-GGPQVTRGDVFTMP-NH2 (product 347751), AC-Lys(MAA) GGPQVTRGDVFTMP-NH2 (product 347241), or MAA-PEG4-lys GGPQVTRGDVFTMP-NH2 (product 347245).

The polypeptide that is conjugated to the monomer may be cyclized or include a cyclic portion. Any suitable method for forming cyclic polypeptide may be employed. For example, an amide linkage may be created by cyclizing the free amino functionality on an appropriate amino-acid side chain and a free carboxyl group of an appropriate amino acid side chain. Also, a di-sulfide linkage may be created between free sulfydryl groups of side chains appropriate amino acids in the peptide sequence. Any suitable technique may be employed to form cyclic polypeptides (or portions thereof). By way of example, methods described in, e.g., WO1989005150 may be employed to form cyclic polypeptides. Head-to-tail cyclic polypeptides, where the polypeptides have an amide bond between the carboxy terminus and the amino terminus may be employed. An alternative to the disulfide bond would be a diselenide bond using two selenocysteines or mixed selenide/sulfide bond, e.g., as described in Koide et al, 1993, Chem. Pharm. Bull. 41(3):502-6; Koide et al., 1993, Chem. Pharm. Bull. 41(9):1596-1600; or Besse and Moroder, 1997, Journal of Peptide Science, vol. 3, 442-453.

Polypeptides may be synthesized as known in the art (or alternatively produced through molecular biological techniques) or obtained from a commercial vendor, such as American Peptide Company, CEM Corporation, or GenScript Corporation. Linkers may be synthesized as known in the art or obtained from a commercial vendor, such as discrete polyethylene glycol (dPEG) linkers available from Quanta BioDesign, Ltd.

An example of a polypolypeptide that may be conjugated to a monomer is a polypeptide that includes KGGNGEPRGDTYRAY (SEQ ID NO:1), which is an RGD-containing sequence from bone sialoprotein with an additional “KGG” sequence added to the N-terminus The lysine (K) serves as a suitable nucleophile for chemical conjugation, and the two glycine amino acids (GG) serve as spacers. Cystine (C), or another suitable amino acid, may alternatively be used for chemical conjugation, depending on the conjugation method employed. Of course, a conjugation or spacer sequence (KGG or CGG, for example) may be present or absent. Additional examples of suitable polypeptides for conjugation with microcarriers (with or without conjugation or spacer sequences) are polypeptides that include NGEPRGDTYRAY, (SEQ ID NO:2), GRGDSPK (SEQ ID NO:3) (short fibronectin) AVTGRGDSPASS (SEQ ID NO:4) (long FN), PQVTRGDVFTMP (SEQ ID NO:5) (vitronectin), RNIAEIIKDI (SEQ ID NO:6) (lamininβ1), KYGRKRLQVQLSIRT (SEQ ID NO:7) (mLMα1 res 2719-2730), NGEPRGDTRAY (SEQ ID NO:8) (BSP-Y), NGEPRGDTYRAY (SEQ ID NO:9) (BSP), KYGAASIKVAVSADR (SEQ ID NO:10) (mLMα1 res2122-2132), KYGKAFDITYVRLKF (SEQ ID NO:11) (mLMγ1 res 139-150), KYGSETTVKYIFRLHE (SEQ ID NO:12) (mLMγ1 res 615-627), KYGTDIRVTLNRLNTF (SEQ ID NO:13) (mLMγ1 res 245-257), TSIKIRGTYSER (SEQ ID NO:14) (mLMγ1 res 650-261), TWYKIAFQRNRK (SEQ ID NO:15) (mLMα1 res 2370-2381), SINNNRWHSIYITRFGNMGS (SEQ ID NO:16) (mLMα1 res 2179-2198), KYGLALERKDHSG (SEQ ID NO:17) (tsp1 RES 87-96), or GQKCIVQTTSWSQCSKS (SEQ ID NO:18) (Cyr61 res 224-240).

In some embodiments, the peptide comprises KGGK⁴DGEPRGDTYRATD¹⁷ (SEQ ID NO:19), where Lys⁴ and Asp¹⁷ together form an amide bond to cyclize a portion of the polypeptide; KGGL⁴EPRGDTYRD¹³ (SEQ ID NO:20), where Lys⁴ and Asp¹³ together form an amide bond to cyclize a portion of the polypeptide; KGGC⁴NGEPRGDTYRATC¹⁷ (SEQ ID NO:21), where Cys⁴ and Cys¹⁷ together form a disulfide bond to cyclize a portion of the polypeptide; KGGC⁴EPRGDTYRC¹³ (SEQ ID NO:22), where Cys⁴ and Cys¹³ together form a disulfide bond to cyclize a portion of the polypeptide, or KGGAVTGDGNSPASS (SEQ ID NO:23).

In embodiments, the polypeptide may be acetylated or amidated or both. While these examples are provided, those of skill in the art will recognize that any peptide or polypeptide sequence may be conjugated to a microcarrier as described herein.

In yet another embodiment, the peptide polymer surface composition may contain multiple peptide sequences. These sequences may be directed toward the adhesion of either a single cell type or to enable multiple cell types to adhere to the same microcarrier beads.

7. Incubating Cells in Culture Media Having Microcarriers

Microcarriers as described herein may be used in any suitable cell culture system. Typically microcarriers and cell culture media are placed in a suitable cell culture article and the microcarriers are stirred or mixed in the media. Suitable cell culture articles include bioreactors, such as the WAVE BIOREACTOR® (Invitrogen), single and multi-well plates, such as 6, 12, 96, 384, and 1536 well plates, jars, petri dishes, flasks, multi-layered flasks, beakers, plates, roller bottles, tubes, bags, membranes, cups, spinner bottles, perfusion chambers, bioreactors, CellSTACK® culture chambers(Corning Incorporated) and fermenters.

A cell culture article housing culture media containing a microcarrier described above may be seeded with cells. The microcarrier employed may be selected based on the type of cell being cultured. The cells may be of any cell type. For example, the cells may be connective tissue cells, epithelial cells, endothelial cells, hepatocytes, skeletal or smooth muscle cells, heart muscle cells, intestinal cells, kidney cells, or cells from other organs, stem cells, islet cells, blood vessel cells, lymphocytes, cancer cells, primary cells, cell lines, or the like. The cells may be mammalian cells, preferably human cells, but may also be non-mammalian cells such as bacterial, yeast, or plant cells.

In numerous embodiments, the cells are stem cells which, as generally understood in the art, refer to cells that have the ability to continuously divide (self-renewal) and that are capable of differentiating into a diverse range of specialized cells. In some embodiments, the stem cells are multipotent, totipotent, or pluripotent stem cells that may be isolated from an organ or tissue of a subject. Such cells are capable of giving rise to a fully differentiated or mature cell types. A stem cell may be a bone marrow-derived stem cell, autologous or otherwise, a neuronal stem cell, or an embryonic stem cell. A stem cell may be nestin positive. A stem cell may be a hematopoeietic stem cell. A stem cell may be a multi-lineage cell derived from epithelial and adipose tissues, umbilical cord blood, liver, brain or other organ. In various embodiments, the stem cells are pluripotent stem cells, such as pluripotent embryonic stem cells isolated from a mammal. Suitable mammals may include rodents such as mice or rats, primates including human and non-human primates. In various embodiments, the microcarrier with conjugated polypeptide supports undifferentiated culture of embryonic stem cells for 5 or more passages, 7 or more passages, or 10 or more passages. Typically stems cells are passaged to a new surface after they reach about 75% confluency. The time for cells to reach 75% confluency is dependent on media, seeding density and other factors as know to those in the art.

Because human embryonic stem cells (hESC) have the ability to grown continually in culture in an undifferentiated state, the hESC for use with microcarriers as described herein may be obtained from an established cell line. Examples of human embryonic stem cell lines that have been established include, but are not limited to, H1, H7, H9, H13 or H14 (available from WiCell established by the University of Wisconsin) (Thompson (1998) Science 282:1145); hESBGN-01, hESBGN-02, hESBGN-03 (BresaGen, Inc., Athens, Ga.); HES-1, HES-2, HES-3, HES-4, HES-5, HES-6 (from ES Cell International, Inc., Singapore); HSF-1, HSF-6 (from University of California at San Francisco); I 3, I 3.2, I 3.3, I 4, I 6, I 6.2, J 3, J 3.2 (derived at the Technion-Israel Institute of Technology, Haifa, Israel); UCSF-1 and UCSF-2 (Genbacev et al., Fertil. Steril. 83(5):1517-29, 2005); lines HUES 1-17 (Cowan et al., NEJM 350(13):1353-56, 2004); and line ACT-14 (Klimanskaya et al., Lancet, 365(9471):1636-41, 2005). Embryonic stem cells may also be obtained directly from primary embryonic tissue. Typically this is done using frozen in vitro fertilized eggs at the blastocyst stage, which would otherwise be discarded.

Other sources of pluripotent stem cells include induced primate pluripotent stem (iPS) cells. iPS cells refer to cells, obtained from a juvenile or adult mammal, such as a human, that are genetically modified, e.g., by transfection with one or more appropriate vectors, such that they are reprogrammed to attain the phenotype of a pluripotent stem cell such as an hESC. Phenotypic traits attained by these reprogrammed cells include morphology resembling stem cells isolated from a blastocyst as well as surface antigen expression, gene expression and telomerase activity resembling blastocyst derived embryonic stem cells. The iPS cells typically have the ability to differentiate into at least one cell type from each of the primary germ layers: ectoderm, endoderm and mesoderm. The iPS cells, like hESC, also form teratomas when injected into immuno-deficient mice, e.g., SCID mice. (Takahashi et al., (2007) Cell 131(5):861; Yu et al., (2007) Science318:5858).

To maintain stem cells in an undifferentiated state it may be desirable to minimize non-specific interaction or attachment of the cells with the surface of the microcarrier, while obtaining selective attachment to the polypeptide(s) attached to the surface. The ability of stem cells to attach to the surface of a microcarrier without conjugated polypeptide may be tested prior to conjugating polypeptide to determine whether the microcarrier provides for little to no non-specific interaction or attachment of stem cells. Once a suitable microcarrier has been selected, cells may be seeded in culture medium containing the microcarriers.

Prior to seeding cells, the cells, regardless or cell type, may be harvested and suspended in a suitable medium, such as a growth medium in which the cells are to be cultured once seeded. For example, the cells may be suspended in and cultured in a serum-containing medium, a conditioned medium, or a chemically-defined medium. As used herein, “chemically-defined medium” means cell culture media that contains no components of unknown composition. Chemically defined cell culture media may, in various embodiments, contain no proteins, hydrosylates, or peptides of unknown composition. In some embodiments, chemically defined media contains polypeptides or proteins of known composition, such as recombinant growth hormones. Because all components of chemically-defined media have a known chemical structure, variability in culture conditions and thus variability in cell response can be reduced, increasing reproducibility. In addition, the possibility of contamination is reduced. Further, the ability to scale up is made easier due, at least in part, to the factors discussed above. Chemically defined cell culture media are commercially available from Invitrogen (Invitrogen Corporation, 1600 Faraday Avenue, PO Box 6482, Carlsbad, Calif. 92008) as STEM PRO, a fully serum- and feeder-free (SFM) specially formulated from the growth and expansion of embryonic stem cells, Xvivo (Lonza), and Stem Cell Technologies, Inc. as mTeSR™1 maintenance media for human embryonic stem cells.

One or more growth or other factors may be added to the medium in which cells are incubated with the microcarriers conjugated to polypeptide. The factors may facilitate cellular proliferation, adhesion, self-renewal, differentiation, or the like. Examples of factors that may be added to or included in the medium include muscle morphogenic factor (MMP), vascular endothelium growth factor (VEGF), interleukins, nerve growth factor (NGF), erythropoietin, platelet derived growth factor (PDGF), epidermal growth factor (EGF), activin A (ACT) such as activin A, hematopoietic growth factors, retinoic acid (RA), interferons, fibroblastic growth factors, such as basic fibroblast growth factor (bFGF), bone morphogenetic protein (BMP), peptide growth factors, heparin binding growth factor (HBGF), hepatocyte growth factor, tumor necrosis factors, insulin-like growth factors (IGF) I and II, transforming growth factors, such as transforming growth factor-β1 (TGFβ1), and colony stimulating factors.

The cells may be seeded at any suitable concentration. Typically, the cells are seeded at about 10,000 cells/cm² of microcarrier to about 500,000 cells/cm². For example, cells may be seeded at about 50,000 cells/cm² of substrate to about 150,000 cells/cm². However, higher and lower concentrations may readily be used. The incubation time and conditions, such as temperature, CO₂ and O₂ levels, growth medium, and the like, will depend on the nature of the cells being cultured and can be readily modified. The amount of time that the cells are cultured with the microcarriers may vary depending on the cell response desired.

The cultured cells may be used for any suitable purpose, including (i) obtaining sufficient amounts of undifferentiated stem cells cultured on a synthetic surface in a chemically defined medium for use in investigational studies or for developing therapeutic uses, (ii) for investigational studies of the cells in culture, (iii) for developing therapeutic uses, (iv) for therapeutic purposes, (v) for studying gene expression, e.g. by creating cDNA libraries, (vi) for studying drug and toxicity screening, and (vii) the like.

One suitable way to determine whether cells are undifferentiated is to determine the presence of the OCT4 marker. In various embodiments, the undifferentiated stems cells cultured on microcarriers as described herein for 5, 7, or 10 or more passages retain the ability to be differentiated.

In various embodiment, the cultured cells are human mesenchymal stem cells (hMSCs). There exist a large number of potential therapeutic applications for hMSCs and clinical trials have already shown promising results. Some cell therapies involving hMSCs that are either in phase I/II include bone and cartilage repair (phase I/II, Aastrom Biosciences, Mesoblast, Osiris Therapeutics,), GVHD (OsirisTherapeutics), acute myocardial infarction, (Osiris Therapeutics) and congestive heart failure (pilot phase I, Angioblast Systems). For cell based therapy and toxicology, hMSCs have been differentiated into osteoblasts, chondrocytes, adipocytes, neuronal cells, skeletal muscle cells, hepatocytes, and cardiomyocytes. Furthermore, hMSCs have been used as cell-based models of human diseases. hMSCs are advantageous over other stem cells types for several reasons. First, they avoid the ethical issues that surround embryonic stem cell research. Second, repeated studies have found that human MSCs are immuno-privileged, and therefore, represent an advantageous cell type for allogenic transplantation, reducing the risks of rejection and complications of transplantation.

To date, no microcarriers have been used for the serum-free expansion of hMSC in a serum-free, chemically defined medium. There is value in being able to culture hMSC in media where the exact composition is known (chemically defined). Furthermore, there is value in having a purely synthetic peptide-based surface to eliminate shortcomings of animal-derived coatings and media.

The hMSCs may be cultured to about 80% confluence on microcarriers beads in chemically defined serum-free media on micro-carrier beads, and may be maintained in an undifferentiated state for at least one passage. The hMSCs may include undifferentiated multi-potent mesenchymal stem cells, such as adipose derived, human umbilical cord derived, or Human Whartons Jelly Mesenchymal Stem Cells.

In the following, non-limiting examples are presented, which describe various non-limiting embodiments of the microcarriers and methods discussed above.

### EXAMPLES

### Example 1: Immobilization of Initiator on Surface of Microcarrier Base

The thermal initiator 4,4′-azobis(4-cyanovaleric acid) or “ACBA” was immobilized on the surface of an amine functionalized polystyrene bead according to the reaction scheme that follows:

Briefly, ACBA (112 mg, 280 g/mol, 0.4 mmol) and EEDQ (197mg, 247 g/mol, 0.8 mmol) were dissolved in 5 mL of N,N-dimethylformamide (DMF), added to a peptide synthesis vessel and bubbled with nitrogen gas for 10 min. 1 gram of dry amine-functionalized polystyrene microspheres (250-315 um, Rapp Polymere, 1.09 mmol NH2/g) was added to the vessel and the beads were mixed by nitrogen bubbling for 24 hours. The microspheres were filtered washed with DMF and ethanol (5×10 mL each) and dried overnight under vacuum. FTIR, C=O amide, 1667 cm⁻¹.

### Example 2: Grafting Synthetic Polymer to Initiator-Conjugated Microcarrier Base and Conjugating Polypeptide in One Pot

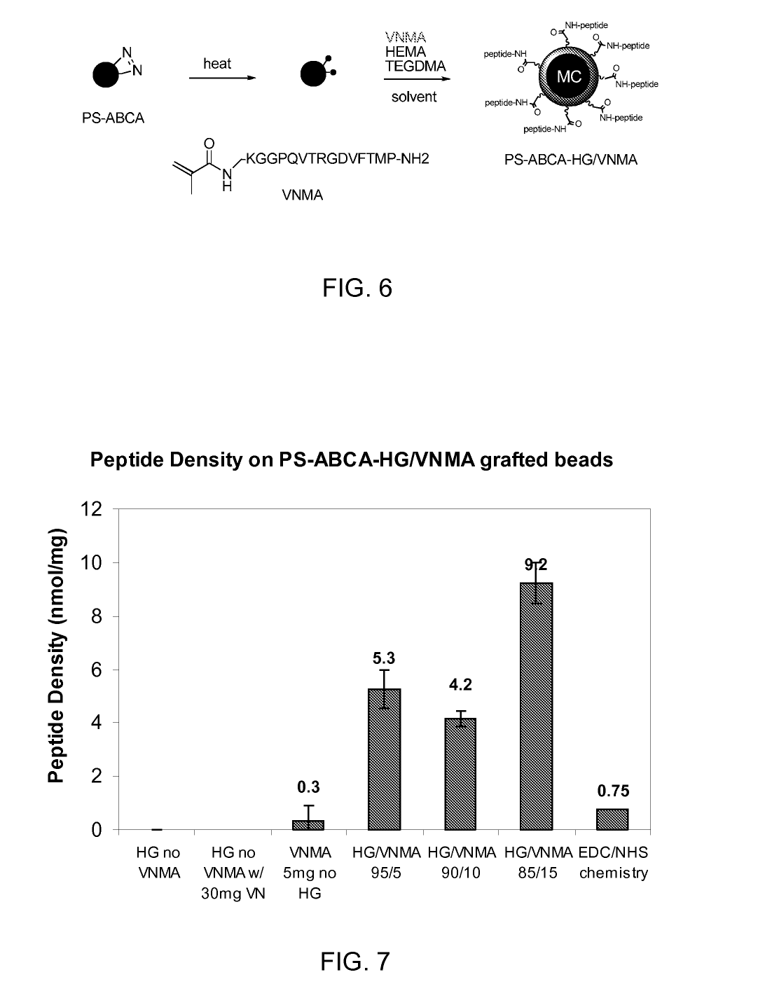

A synthetic polymer layer was grafted to the ACBA-conjugated microspheres described in Example 1, where the polymerization included the addition of a polypeptide to the surface, according to the reaction scheme presented in FIG. 6.

ABCA is covalently attached to the surface using EEDQ activation. The dry initiator bead is re-suspended in a solvent solution containing the acrylate monomers and peptide methacrylate (VNMA) where it is heated to generate free-radicals. The surface initiated polymerization results in grafting of the synthetic polymer and peptide to the surface of the microsphere. Using this new peptide acrylate strategy, the peptide grafting efficiency is significantly improved over the EDC/NHS activation chemistry, such as employed in U.S. Provisional Application Ser. No. 61/229,114, filed on Jul. 28, 2009, having attorney docket number of SP09-221P.

Briefly, 200 mg of ABCA immobilized microspheres (15112-143) were combined with VNMA, HEMA, TEGDMA (in the amounts listed in the Table 1 below) and suspended in 4 mL of methanol in a 15 mL long neck ampoule equipped magnetic stirrer. The ampoule was sealed and the slurry was degassed with nitrogen for 1 hr, followed by mixing at 80° C. for 1 hr. The VNMA grafted microcarriers were aspiration washed with methanol (5×15 mL), transferred to a 15 mL centrifuge tube and washed by rotation on an orbital shaker in the presence of 15 mL of ethanol/water 1:1 for 1 hr. The microcarriers were then rinsed with ethanol (3×5 mL each) and air dried overnight.

TABLE 1
Experimental conditions used in the preparation
of PS-ABCA-VNMA microcarriers.

| Experiment | HEMA | PMA | TEGDMA | Solvent | Comments |
|---|---|---|---|---|---|
| 15112-144A | 200 uL | 0 mg | 6 uL | MeOH (4 mL) | Control 1 |
| | | | | | No VN |
| 15112-144B | 170 uL | 30 mg | 6 uL | MeOH (4 mL) | 85/15 |
| 15112-144C | 190 uL | 10 mg | 6 uL | MeOH (4 mL) | 95/5 |
| 15112-144D | 180 uL | 20 mg | 6 uL | MeOH (4 mL) | 90/10 |
| 15112-144E | 170 uL | 30 mg | 6 uL | MeOH (4 mL) | 85/15 |
| | | of VN | | | control 2 |
| 15112-114F | 0 | 5 mg | 0 | MeOH (4 mL) | |

Examples of conditions used during the preparation of synthetic peptide acrylate microcarriers. Experiments 15112-144A and 15112-144E represent negative controls where no peptide should be present in the grafted polymer (verified by an on-bead BCA assay). Experiment 15112-144F represents grafting of peptide alone without other monomers to generate synthetic polymer. Experiments 15112-144B-D represent varying levels of peptide acrylate in grafting solution.

### Example 3: Peptide Density Estimation

Peptide densities on PS-ABCA-VN microcarriers prepared in accordance with Example 2 were estimated using a modified BCA assay. Briefly, the Interchem BCA working reagent was prepared by adding 1 part of reagent B to 50 parts of reagent A in a 50 mL centrifuge tube. The standard solutions were prepared by serial dilution of a 10 mM Vitronectin solution down to 1 uM. 5-10 mg of dry VN modified microcarriers and base microcarriers were added in duplicate to separate wells of a Corning ultra low attachment (ULA) 24 well plate. 25 ul of each standard solution was also introduced into separate wells of the ULA 24 well plate. To each standard solution and sample was added 800 ul of the BCA working reagent per test well and the plate was incubated for 2 hours at 25° C. (gently mixing the plate every 30 min to re-suspend microcarriers). 750 uL of BCA color developed standard and sample solutions were removed (place pipette tip in corner of well to minimize transfer of beads from sample) and the optical absorbance was read at 562 nm on an UV-Vis spectrophotometer (instrument blanked with PBS). To estimate peptide density, the blank absorbance was subtracted from all others to get net absorbance to generate a standard curve of net absorbance as a function of VN concentration. The linear fit up to 5 mM was used to generate a correlation formula. The absorbance of the base bead (no VN) was subtracted from VN-sample absorbance to get the sample net absorbance. The correlation formula was then used to estimate peptide density in nmol/mg.

The results are presented in FIG. 7. As shown in FIG. 7, the peptide densities ranged from 0.3 to 9.2 nmol/mg. When HEMA and TEGDMA were not present (Experiment 15112-114F) low levels of peptide grafted to the surface compared to when present. As a comparison, the EDC/NHS gives significantly lower peptide densities (0.7 nmol/mg). EDC/NHS grafting was performed as described in U.S. Provisional Application Ser. No. 61/229,114, filed on Jul. 28, 2009, having attorney docket number of SP09-221P, where carboxyethylacrylate (CEA) was used in place of VNMA and a synthetic Vitronectin peptide with a C-terminal lysine was conjugated to carboxylate groups from the CEA via EDC/NHS chemistry.

### Example 4: Bone Marrow Derived Human Mesenchymal Stem Cell Expansion

Bone marrow derived human mesenchymal stem cells (hMSC) were purchased from Millipore Corporation. The cells were gently agitated at 37° C. water bath until almost completely thawed. The cells were added to 0.1% gelatin coated T75 flask containing approximately 25 ml of hMSC expansion medium completed with FGF. The T-25 flask was transferred into an incubator set a 37° C., 5% CO₂, with 95% humidity. Fresh medium was added every alternate day until cells reached 80% confluency by visual inspection under a microscope.

### Example 5: Human Mesenchymal Stem Cell Propagation on PS-ABCA-PMA Microcarriers

At 80% confluency, hMSC were first split using trypsin-EDTA. Cell number was determined using Beckman-Coulter particle counter. 10 mg beads of PS-ABCA-VNMA (vitronectin coated) microcarriers and negative control sample (no VN) were first placed into two wells of a 24 well Costar ULA plate. The beads were aspiration washed with compatible medium before adding cells at 240000 cells per well in 500 μL medium (10 mg of the PS-ABCA-PMA microcarriers have surface area of about 3 wells of a 24 well plate (1.56 cm²). The 24 well plate cell containing microcarriers were transferred into an incubator set a 37° C., 5% CO₂, with 95% humidity. Imaging was done every day after seeding up to 3 days. On day 2, old beads were distributed among three different wells. Approximately, 3 mg of fresh beads were added to old beads in all three wells and cell expansion from old to fresh beads was observed in three different media conditions. On day 3, cells that adhered to the microcarriers were fixed with 4% formaldehyde.

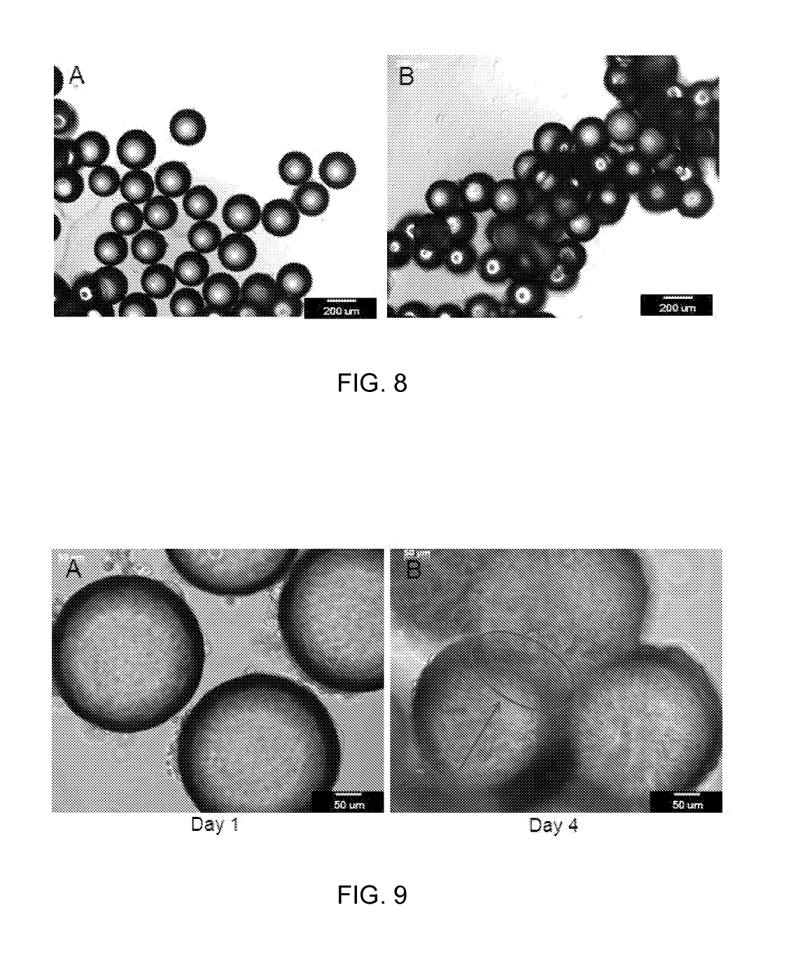

Representative images of hMSCs on PS-ABCA-VNMA microcarriers after 4 days of culture are shown in FIG. 8. In panel A, the microcarriers are from experiment 15112-144A. In panel B, the microcarriers are control VNMA peptide grafted microcarriers (experiment 15112-114A). Due to cell growth and expansion, the VN coated microspheres exhibit a flocking phenomenon while uncoated microsphere did not. No cell attachment is observed on microcarriers without VN adhesion peptide (used as negative control), data not shown.

A magnified view showing clumping is shown in FIG. 9. At day 1 (panel A), no clumping was observed. However, at day 4 (panel B), cell attachment from various microspheres led to clumping. Cell-cell attachment due to cell growth and expansion from one bead to another is shown in circle indicated by arrow (panel B).

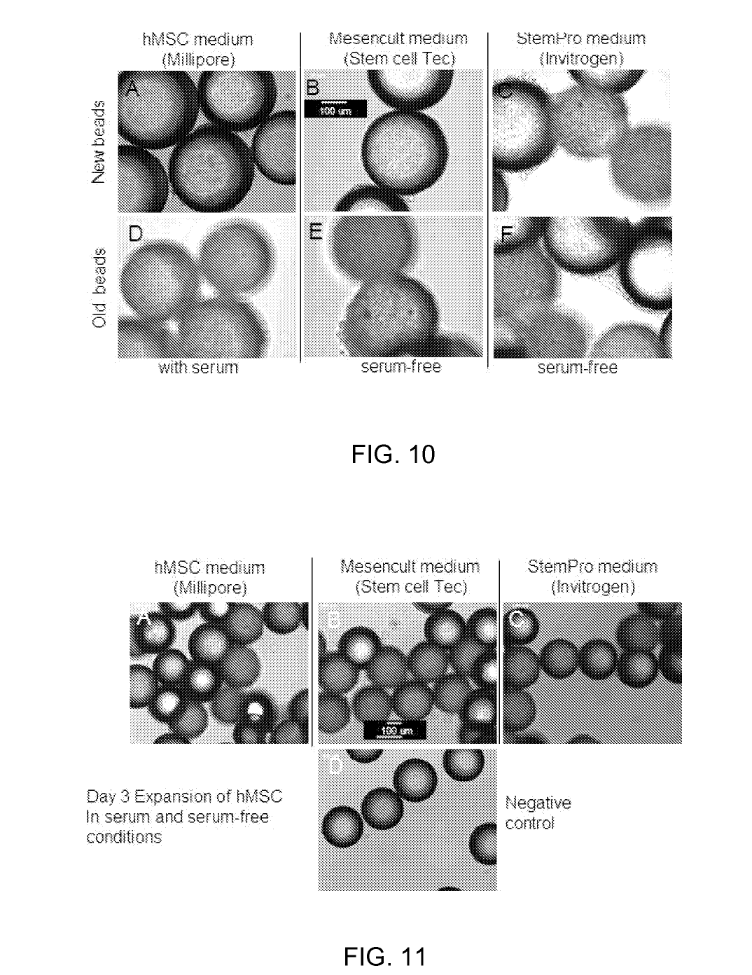

Referring now to FIG. 10, micrograph images of hMSCs on PS-ABCA-VNMA microcarriers after 2 days of culture in serum (A, D) and serum-free conditions (B, C, E, F) are shown. Cell containing microcarriers were split and added to fresh microcarriers containing no cells. The images on top (panels A-C) illustrate a mix of new and old microcarriers after new microcarrier addition. The images on the bottom (panels D-F) illustrate microcarriers where hMSC have grown to over confluency on previously added microcarriers. The cells were cultured on microcarriers generated in accordance with experiment 15112-144B in Example using hMSC medium from Millipore (panels A and D), Mesencult medium from Stem cell Tec (panels B and E), or StemPro medium from Invitrogen (panels C and F). Otherwise, the cells were cultured as described above.

### Example 6: Immunostaining of PS-ABCA-VNMA Microcarriers Containing hMSC

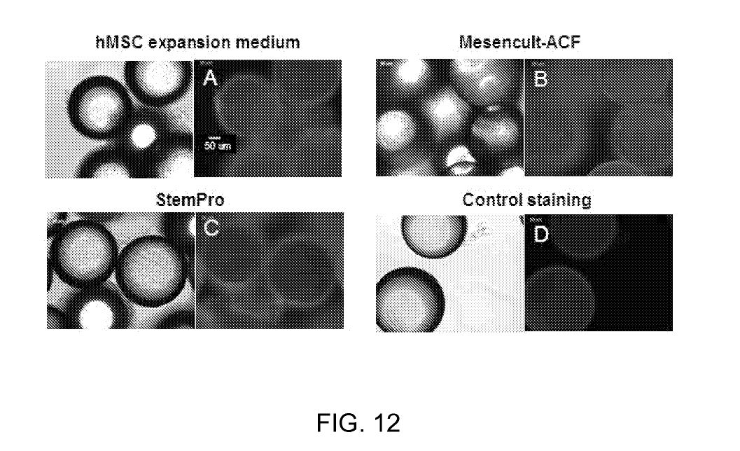

Approximately 6 mg of microcarriers containing fixed and blocked cells suspended overnight in 0.5 mL of the primary antibody H-CAM (CD44) in blocking solution. The microcarriers were then aspiration washed with the blocking solution, followed by addition of a FITC conjugated secondary antibody (0.5 mL). Cell/beads were allowed sit in the dark for 2 hr, followed by aspiration washing. Thereafter, the nuclei of the cells attached to the microcarriers were Hoechst stained. The staining was assessed using a Ziess Axiovert 200M inverted Brightfield/fluorescence microscope using FITC and DAPI channels (FIGS. 11 and 12).

In FIG. 11, micrographs of hMSC on PS-ABCA-VNMA microcarriers after 3 days of culture in serum and serum-free conditions are shown, where the cells were cultured in the medium as shown. hMSC continue to proliferate onto fresh PS-ABCA-VNMA coated microcarriers (experiment 15112-144B microcarriers) in all media tested (serum and serum-free). No cells were present on negative control surface (experiment 15112-144A microcarriers) after 3 days.

In FIG. 12, immunostaining of hMSCs with positive cell marker, mouse anti-H-CAM (CD44) after 3 days of expansion on PS-ABCA-VNMA microcarriers in serum and serum-free conditions are shown. Staining confirmed presence of undifferentiated hMSC on PS-ABCA-VNMA microcarriers (experiment 15112-144B microcarriers). In addition, it was more or less the same in all media tested (serum and serum-free). No cell staining was observed on control surfaces (experiment 15112-144A microcarriers).

Thus, embodiments of SYNTHETIC PEPTIDE (METH)ACRYLATE MICROCARRIERS are disclosed. One skilled in the art will appreciate that the microcarriers and methods described herein can be practiced with embodiments other than those disclosed. The disclosed embodiments are presented for purposes of illustration and not limitation.

What is claimed is:

1. A one-pot process for forming a coated microcarrier with conjugated polypeptide, comprising:
providing an initiator-conjugated microcarrier base having a polymerization initiator conjugated to a microcarrier base;
contacting the initiator-conjugated microcarrier base with one or more monomers, wherein at least one of the monomers is conjugated to a polypeptide; and
activating the initiator to initiate polymerization and to graft the polymer from the base via the initiator or a remnant thereof.

2. A process according to claim 1, wherein the monomer conjugated to the polypeptide is capable of polymerizing via a free-radical, cationic or redox reaction.

3. A process according to claim 1, wherein the polypeptide is conjugated to the monomer via a linker comprising poly(ethylene-oxide) having between 1 and 10 repeating ethylene-oxide units.

4. A process according to claim 1, wherein the polypeptide is a vitronectin polypeptide.

5. A process according to claim 4, wherein the polypeptide comprises an amino acid sequence of PQVTRGDVFTMP (SEQ ID NO: 5).

6. A process according to claim 1, wherein the monomer conjugated to the polypeptide is a (meth)acrylate monomer.

7. A process according to claim 1, wherein contacting the initiator-conjugated microcarrier base with one or more monomers comprises contacting the initiator-conjugated microcarrier with the monomer conjugated to the polypeptide, a cross-linking monomer, and a hydrophilic monomer capable of polymerizing with the cross-linking monomer and the monomer conjugated to the polypeptide.

8. A process according to claim 7, wherein the cross-linking monomer is a (meth)acrylate cross-linking monomer.

9. A process according to claim 7, wherein the hydrophilic monomer is a (meth)acrylate hydrophilic monomer.

10. A process according to any of claim 1, wherein the initiator is selected from the group consisting of 4,4′-Azobis-(4-cyanopentanoic acid), 4-(3-hydridodiethylsilyl)propyloxybenzophone, (3-(2-bromoisobutyryl)propyl) diethylhydridosilane, and 2-bromo-isobutyryl bromide.

11. A coated microcarrier having a conjugated polypeptide prepared by the process of claim 1.

12. A coated microcarrier having a conjugated polypeptide, comprising:
a microcarrier base;
a polymerization initiator, or a remnant thereof, conjugated to the microcarrer base;
a polymer coating conjugated to the microcarrier base via the initiator or remnant thereof; and
a polypeptide conjugated to the polymer coating, wherein prior to being conjugated to the polymer coating, the polypeptide is conjugated to a monomer that reacts to form a part of the polymer coating.

13. A coated microcarrier having a conjugated polypeptide according to claim 12, wherein the polypeptide is a vitronectin polypeptide.

14. A coated microcarrier having a conjugated polypeptide according to claim 12, wherein the polymer coating is a swellable (meth)acrylate coating.

15. A coated microcarrier having a conjugated polypeptide according to claim 12, wherein the polypeptide is conjugated to the monomer via a linker, wherein the linker is a poly(ethylene-oxide) linker having between 1 and 10 repeating ethylene-oxide units.

16. A coated microcarrier according to claim 15, wherein the poly(ethylene-oxide) linker has 4 repeating ethylene-oxide units.

17. A method for culturing cells, comprising:
contacting the cells with a microcarrier according to any of claims 12 in a cell culture medium, and
culturing the cells in the medium.

18. A method according to claim 17, wherein the cells are stem cells.

19. A method according to claim 17, wherein the cells are human bone marrow mesenchymal stem cells.

20. A method according to claim 17, wherein the cell culture medium is a chemically-defined cell culture medium.


SEQUENCE LISTING (23 sequences)

SEQ ID NO: 1 - protein, 15 aa, Artificial Sequence
  Synthetic polypeptide
KGGNGEPRGDTYRAY

SEQ ID NO: 2 - protein, 12 aa, Artificial Sequence
  Synthetic polypeptide
NGEPRGDTYRAY

SEQ ID NO: 3 - protein, 7 aa, Artificial Sequence
  Synthetc polypeptide
GRGDSPK

SEQ ID NO: 4 - protein, 12 aa, Artificial Sequence
  Synthetic polypeptide
AVTGRGDSPASS

SEQ ID NO: 5 - protein, 12 aa, Artificial Sequence
  Synthetic polypeptide
PQVTRGDVFTMP

SEQ ID NO: 6 - protein, 10 aa, Artificial Sequence
  Synthetic polypeptide
RNIAEIIKDI

SEQ ID NO: 7 - protein, 15 aa, Artificial Sequence
  Synthetic polypeptide
KYGRKRLQVQLSIRT

SEQ ID NO: 8 - protein, 11 aa, Artificial Sequence
  Synthetic polypeptide
NGEPRGDTRAY

SEQ ID NO: 9 - protein, 12 aa, Artificial Sequence
  Synthetic polypeptide
NGEPRGDTYRAY

SEQ ID NO: 10 - protein, 15 aa, Artificial Sequence
  Synthetic polypeptide
KYGAASIKVAVSADR

SEQ ID NO: 11 - protein, 15 aa, Artificial Sequence
  Synthetic polypeptide
KYGKAFDITYVRLKF

SEQ ID NO: 12 - protein, 16 aa, Artificial Sequence
  Synthetic polypeptide
KYGSETTVKYIFRLHE

SEQ ID NO: 13 - protein, 16 aa, Artificial Sequence
  Synthetic polypeptide
KYGTDIRVTLNRLNTF

SEQ ID NO: 14 - protein, 12 aa, Artificial Sequence
  Synthetic polypeptide
TSIKIRGTYSER

SEQ ID NO: 15 - protein, 12 aa, Artificial Sequence
  Synthetic polypeptide
TWYKIAFQRNRK

SEQ ID NO: 16 - protein, 20 aa, Artificial Sequence
  Synthetic polypeptide
SINNNRWHSIYITRFGNMGS

SEQ ID NO: 17 - protein, 13 aa, Artificial Sequence
  Synthetic polypeptide
KYGLALERKDHSG

SEQ ID NO: 18 - protein, 17 aa, Artificial Sequence
  Syntehtic polypeptide
GQKCIVQTTSWSQCSKS

SEQ ID NO: 19 - protein, 17 aa, Artificial Sequence
  Synthetic polypeptide
KGGKDGEPRGDTYRATD

SEQ ID NO: 20 - protein, 13 aa, Artificial Sequence
  Synthetic polypeptide
KGGLEPRGDTYRD

SEQ ID NO: 21 - protein, 17 aa, Artificial Sequence
  Synthetic polypeptide
KGGCNGEPRGDTYRATC

SEQ ID NO: 22 - protein, 13 aa, Artificial Sequence
  Synthetic polypeptide
KGGCEPRGDTYRC

SEQ ID NO: 23 - protein, 15 aa, Artificial Sequence
  Synthetic polypeptide
KGGAVTGDGNSPASS